(19) United States
(12) Patent Application Publication
(10) Pub. No.: US 2010/0300450 A1
(43) Pub. Date: Dec. 2, 2010

(54) NASAL AIRWAY MANAGEMENT DEVICE WITH INFLATABLE SUPRAGLOTTIC LARYNGEAL CUFF

(75) Inventor: Viachaslau M. Barodka, Baltimore, MD (US)

Correspondence Address: VENABLE LLP, P.O. BOX 34385, WASHINGTON, DC, 20043-9998, US

(73) Assignee: The Johns Hopkins University, Baltimore, MD (US)

(21) Appl. No.: 12/789,586

(22) Filed: May 28, 2010

Related U.S. Application Data
(60) Provisional application No. 61/181,731, filed on May 28, 2009.

(51) Int. Cl. A61M 16/00

(52) U.S. Cl. 128/207.18

(57) ABSTRACT

An airway management device such as, for example, an nasal airway management device with inflatable supraglottic laryngeal cuff is provided. The airway management device includes an airway tube, an inflatable cuff, and an inflation passage. The airway tube defines a lumen extending between a proximal end and a distal end. At least the distal end of the airway tube is configured to be inserted through a patient's nasopharyngeal passageway. The inflatable cuff is attached at or near the distal end of the airway tube and defines an opening fluidly coupled with the lumen of the airway tube. The inflation passage may extend along the airway tube and may be coupled to the inflatable cuff to allow inflation and deflation thereof. When in a deflated state the inflatable cuff may be inserted through the patient's nasopharyngeal passageway. When in an inflated state the inflatable cuff may expand to form a seal around the patient's supraglottic laryngeal inlet. A method for utilizing the airway management device to manage a patient's airway may also be provided.

### CROSS-REFERENCE TO RELATED APPLICATIONS

This application claims priority to U.S. Provisional Application No. 61/181,731 filed May 28, 2009, the entire content of which is hereby incorporated by reference.

### BACKGROUND

1. Field of Invention

The invention relates generally to airway management devices and, more particularly, to a nasal airway management device with inflatable supraglottic laryngeal cuff.

2. Discussion of Related Art

Any discussion of the related art throughout the specification should in no way be considered as an admission that such art is widely known or forms part of general knowledge in the field.

Maintaining and securing a patient's airway and providing adequate ventilation when necessary are core principles to ensure oxygen delivery in all human beings. Controlling a patient's airway is the first and most important principle in basic life support and advanced cardiac life support algorithms. Humans cannot survive the interruption of oxygen delivery to the body for more than 5-10 minutes. This puts enormous pressure on ensuring timely and reliable means of airway control in emergency situations. Anesthetized patients, sedated patients, critically ill patients and coding patients, for example, often can not maintain an airway or adequately breath on their own and almost always require assisted ventilation. Accordingly, airway devices are typically placed in a patient's airway by trained professionals in order to assist with ventilation or to maintain and protect the airway. These devises may include, for example, oral and nasal airways, laryngeal mask airways (LMAs), and endotracheal tubes. These devices, however, have several disadvantages. For example, highly trained healthcare personnel and special instrumentation are typically required for the placement of advanced airway devices such as LMAs and endotracheal tubes. Moreover, sedation or general anesthesia are required during placement and use of LMAs and endotracheal tubes. Other devices such as, for example, nasal and oral airways and external face masks, while less invasive, nevertheless provide limited or no ability to provide assisted ventilation.

There is a need for quick, simple and reliable airway management device to provide airway control and assisted ventilation in, for example but not limited to, emergency situations.

### SUMMARY

In an embodiment of the invention, an airway management device is provided. The airway management device according to an embodiment of the invention includes an airway tube, an inflatable cuff, and an inflation passage. The airway tube defines a lumen extending between a proximal end and a distal end. At least the distal end of the airway tube may be configured to be inserted through a patient's nasopharyngeal passageway. The inflatable cuff may be attached at or near the distal end of the airway tube. The inflatable cuff may define an opening fluidly coupled with the lumen of the airway tube. The inflation passage may extend along the airway tube and may be coupled to the inflatable cuff to allow inflation and deflation thereof. When in a deflated state the inflatable cuff may be inserted through the patient's nasopharyngeal passageway. When in an inflated state the inflatable cuff may expand to form a seal around the patient's supraglottic laryngeal inlet.

In another embodiment of the invention, a method for utilizing the airway management device to manage a patient's airway may also be provided. The method according to an embodiment of the invention includes inserting the distal end of the airway management device through the patient's nasopharyngeal passageway to a position in the laryngopharynx with the inflatable cuff in the deflated state. The inflatable cuff may include a flexible material densely packed about the distal end of the airway tube when the inflatable cuff is in the deflated state to facilitate insertion of the distal end of the airway tube through the patient's nasopharyngeal passageway. The method may further include inflating the cuff until the supraglottic laryngeal inlet is substantially sealed, and artificial ventilating the patient via the opening defined by the inflatable cuff.

Further features and advantages, as well as the structure and operation of various embodiments of the invention, are described in detail below with reference to the accompanying drawings.

### BRIEF DESCRIPTION OF THE DRAWINGS

The foregoing and other features and advantages of the invention will be apparent from the following, more particular description of some example embodiments of the invention, as illustrated in the accompanying drawings. Unless otherwise indicated, the accompanying drawing figures are not to scale. Several embodiments of the invention will be described with respect to the following drawings, in which like reference numerals represent like features throughout the figures, and in which:

FIG. 1 depicts a perspective view of a nasal airway management device having an inflatable supraglottic laryngeal cuff in a deflated state according to an embodiment of the invention;

FIG. 2 depicts a perspective view of the nasal airway management device of FIG. 1 with the inflatable supraglottic laryngeal cuff in an inflated state according to an embodiment of the invention;

FIG. 3 depicts the nasal airway management device of FIGS. 1 and 2 in an inflated state in a patient's laryngopharynx after insertion through the nasopharyngeal passageway;

FIG. 4 depicts a perspective view of a nasal airway management device according to another embodiment of the invention;

FIG. 5 depicts a perspective view of a nasal airway management device having an inflatable supraglottic laryngeal cuff and a second positioning cuff in an inflated state according to another embodiment of the invention; and

FIG. 6 depicts the nasal airway management device of FIG. 5 in an inflated state in a patient's laryngopharynx after insertion through the nasopharyngeal passageway.

### DETAILED DESCRIPTION

Some embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the invention. All references cited herein are incorporated by reference as if each had been individually incorporated.

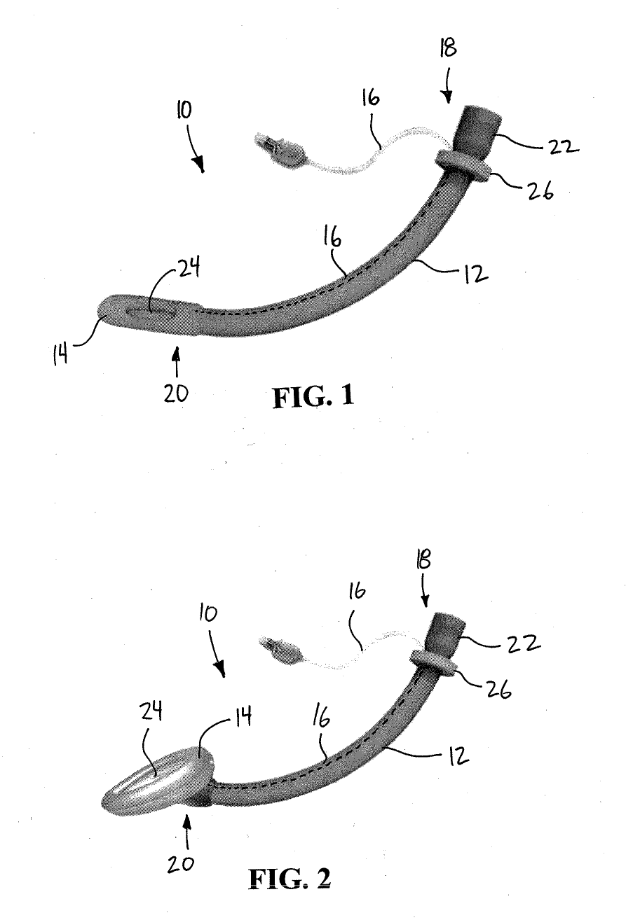

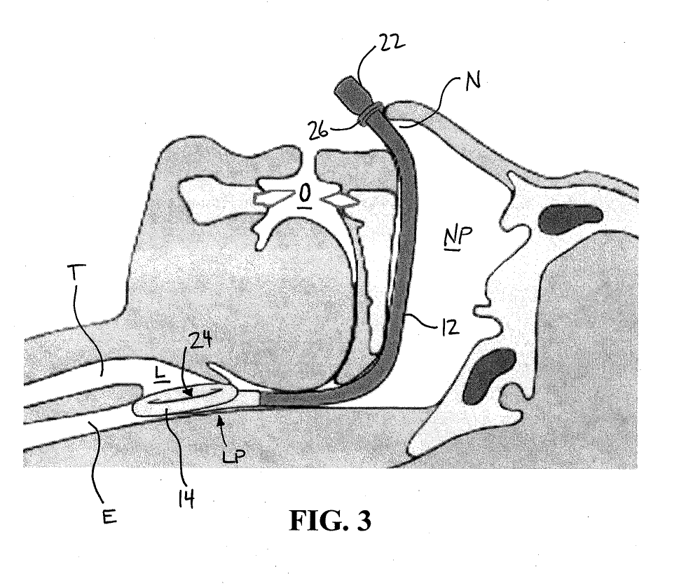

FIG. 1 depicts a perspective view of a nasal airway management device 10 having an inflatable cuff 14 in a deflated state according to an embodiment of the invention. The airway management device 10 includes an airway tube 12, the inflatable cuff 14, and an inflation passage 16. The airway tube 12 defines a lumen (not shown) extending between a proximal end 18 and a distal end 20. The proximal end 18 of the airway tube 12 may include a connector portion 22 configured to be coupled to a ventilator, a ventilation bag, or an anesthetic circuit (not shown). The airway tube 12, particularly the distal end 20, is configured for insertion through a patient's nostril N and nasopharyngeal passageway NP to a position in the laryngopharynx LP (see FIG. 3). The airway tube 12 may be formed from a variety of suitable materials such as, for example but not limited to, polyvinylchloride (PVC), although other materials are possible so long as they are body tolerated and constructed to provide enough rigidity to prevent collapse, twisting, kinking and/or buckling of the tube in the nasal cavity and in the pharynx. The tube 12 should also be flexible enough to avoid causing nasal trauma during insertion and use.

The inflatable cuff 14 is attached at the distal end 20 of the airway tube 12 and may be arranged to be inflated and deflated via the inflation passage 16. The inflation passage 16 may extend along the airway tube 12 and may be coupled to the inflatable cuff 14 to allow inflation and deflation thereof. The inflation passage 16 may include a pilot tube attached along an external surface of the airway tube 12, along an internal surface of the lumen of the airway tube 12, or at least partly integrally formed within a wall of the airway tube 12. An adjustable annular flange 26 may be moveably disposed on an outer periphery of the airway tube 12 near the proximal end 18 to help stabilize and hold the airway tube 12 in place at the patient's nostril N.

The inflatable cuff 14 attached at the distal end 20 of the airway tube 12 may include thin, flexible and compliant material such as, for example, PVC, although other materials are possible. When in a deflated state such as, for example, as shown in the embodiment depicted in FIG. 1, the inflatable cuff 14 may be sized and configured to be inserted through the patient's nasopharyngeal passageway NP (see FIG. 3). For example, the inflatable cuff 14 may be densely packed about the distal end 20 of the airway tube 12 in a such way that it is approximately continuous with the outer wall of the airway tube 12. The deflated cuff 14 may, for example, have an outer cuff dimension (e.g., an outer diameter) that is substantially equal to or less than an outer tube dimension (e.g., an outer diameter) of the airway tube 12 to facilitate insertion via the patient's nostril N and nasopharyngeal passageway NP. In an embodiment, the densely packed inflatable cuff 14 may form a substantially conical shape extending from the distal end 20 of the airway tube 12. The deflated cuff 14 may define, for example, a round, smooth elongated tip with a gradually increasing diameter to facilitate atraumatic insertion of the device 10. The inflatable cuff 14 defines an opening 24 fluidly coupled with the lumen of the airway tube 12 to allow passage of air or other gaseous substance. In an embodiment not shown in greater detail herein, the inflatable cuff 14 may comprise an outer portion and an inner portion. The outer portion may be formed from a relatively thick and semi-rigid material such as, for example, of the same thickness and rigidity as the airway tube 12. The inner portion may be of a relatively thinner and more flexible material densely packed inside the outer portion about the distal end 20 of the airway tube 12 when the inflatable cuff 14 is in the deflated state to facilitate insertion of the distal end 20 of the airway tube 12 through the patient's nasopharyngeal passageway NP. Once inflated, the outer portion unfolds and forms a semi-rigid base wider then the diameter of the airway tube 12 against posterior wall of the laryngopharynx forming a base and structural support for the inner portion of the cuff 14. The inflated inner portion the cuff 14 expands on the edges of the semi-rigid outer portion and forms a final shape to provide a seal of the supraglottic laryngeal inlet.

FIG. 2 depicts a perspective view of the nasal airway management device 10 of FIG. 1 with the inflatable cuff 14 in an inflated state according to an embodiment of the invention. When in the inflated state, the inflatable cuff 14 may expand to a shape and size configured to form a seal around the patient's supraglottic laryngeal inlet L (see FIG. 3). For example, the inflated cuff 14 may form a substantially elliptical or oval structure circumscribing the opening 24 and disposed at an angle to a longitudinal axis of the airway tube 12. In this regard, the cuff 14 can function as a nasally-inserted supraglottic laryngeal mask, allowing ventilation of the patient while the patient's oral cavity O remains free of any airway devices. FIG. 3, for example, depicts a side cross-sectional view of a patient's head with the inflatable cuff 14 of the device 10 positioned in the patient's laryngopharynx LP in an inflated state after insertion through the nostril N and nasopharyngeal passageway NP. The inflatable cuff 14 seals the larynx and trachea T from the esophagus E and pharynx and allows a healthcare provider to deliver PPV (positive pressure ventilation), CPAP (continuous positive airway pressure) or PEEP (positive end expiratory pressure) to the patient via the device 10. Thus, once inflated, the cuff 14 may be in the form of, for example, a triangular pyramid occupying the laryngopharynx LP, the space between the glottic opening and choana (i.e., the anatomical opening of the nasal airway passage in the nasopharynx). Inflation of the cuff 14 lifts the epiglottis away from the glottic opening to prevent it from obstructing the airway (see FIG. 3). Alternatively, when the inflatable cuff 14 is positioned in the patient's laryngopharynx LP in the deflated state, the device 10 may serve to simply maintain patency of the patient's airway and allow spontaneous ventilation via the opening 24.

As shown in the embodiment depicted in FIGS. 1-3, the airway tube 12 may include a curved shaped such as, for example, a U-shape to mimic nasal airway anatomy and add a second dimension to allow only one position for insertion. Other positioning structures and/or indicator markings are, however, possible to ensure correct positioning of the cuff 14 adjacent to the supraglottic laryngeal inlet L. The device 10 may be offered in different sizes to fit different patients. Before placement, the cuff 14 should be in the deflated state. Lubrication jelly, possibly including local anesthetic, may be applied to the cuff 14 and along the outer wall of the airway tube 12 from the distal end 20 to assist with gentle insertion through the patient's nostril N and nasopharyngeal passageway NP. Local anesthetic spray may also be applied to the patient's nostril N. Once inserted, the cuff 14 may be inflated via the inflation passage 16 by, for example, a 10-20 cc syringe filled with air or another suitable gas or liquid substance. The inflated cuff 14 will make the form of a laryngeal mask providing the seal between larynx and the rest of pharynx and esophagus E. Inflation of the cuff 14 may protect the vocal cords and airway and allow the delivery of PPV, CPAP or PEEP assisted ventilation.

When PPV, CPAP, or PEEP is no longer needed, or if the patient should undergo unassisted spontaneous breathing trials such that simple maintenance the patency of the airway remains, the device 10 can be left in place with cuff 14 deflated. In this case, the device 10 can serve as a conventional nasal airway. Nasal airways provide minimal stimulation to the patient and are extremely well tolerated compared to endotracheal tubes or LMAs. In addition the patient would be able to talk. At any time, the cuff 14 could be inflated again if there is a need to resume assisted ventilation.

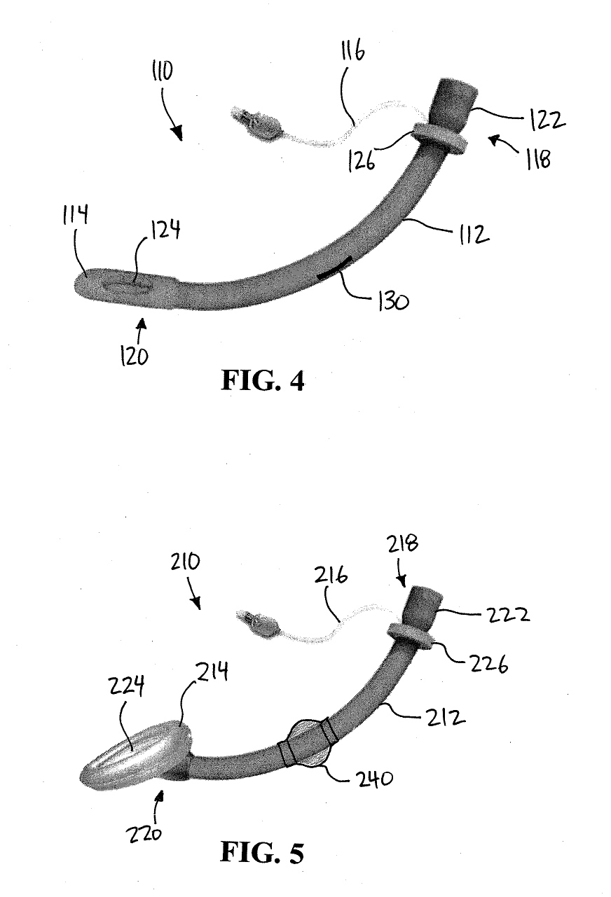

FIG. 4 depicts a perspective view of a nasal airway management device 110 according to another embodiment of the invention. Airway management device 110 is substantially the same as device 10, described above, except that device 110 may include a probe 130 embedded in, or attached to, the wall of the airway tube 112. The probe 130 may be positioned at a point along the airway tube 112 such that, when the airway tube 112 is inserted through the nasopharyngeal passageway NP, the probe 130 is disposed in the nasopharynx to monitor the patient. The probe 130 may include, for example but not limited to, a temperature measurement device such as, for example but not limited to, a thermocouple, a thermistor, or a resistance temperature detector (RTD). The probe 130 could also include, for example, but not limited to a pH probe or a local tissue CO2 measurement probe which would reflect tissue perfusion and adequacy of ventilation, respectively. The device 110 could also include a small passage with opening near distal end for air sampling (not shown). Air sampling is needed for the detection of CO2 and quantification of respiratory gases. Sampling may be done at the proximal end when anesthesia circuit is connected.

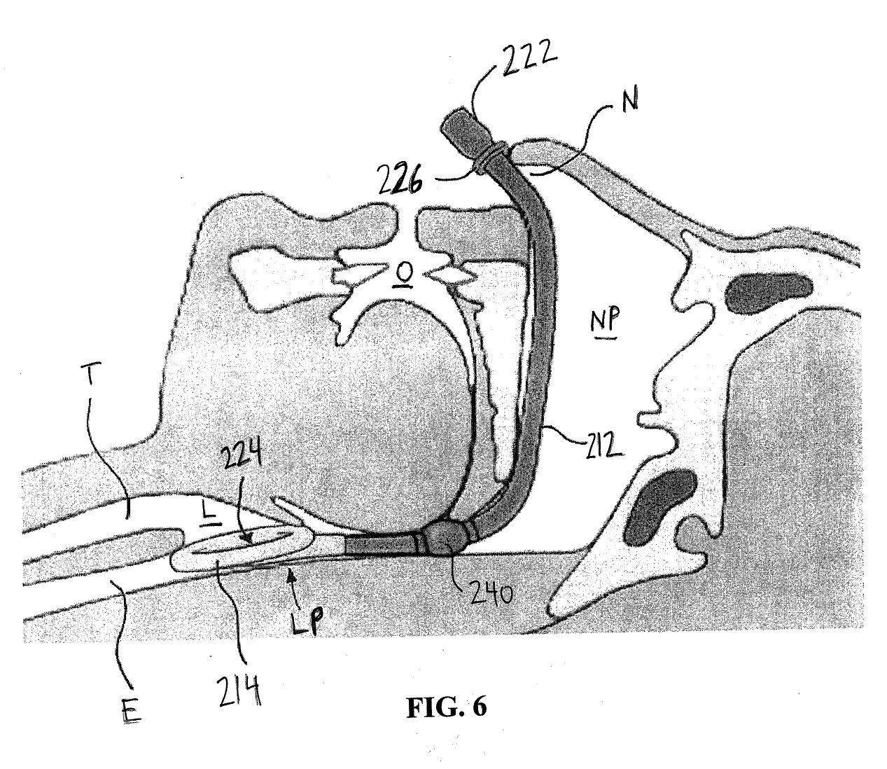

FIG. 5 depicts a perspective view of a nasal airway management device 210 having an inflatable cuff 214 and a second positioning cuff 240 in an inflated state according to another embodiment of the invention. Airway management device 210 is substantially the same as device 10, described above, except that device 210 may include the second inflatable cuff 240 at a position along the airway tube 212 between the proximal and distal ends 218, 220, respectively. The second inflatable cuff 240 may be configured to be inflated in order to fix and hold the airway tube 212 in position patient's nasopharyngeal passageway NP. When the device 210 is inserted with both cuffs 214, 240 in the deflated state, the second cuff 240 should be positioned on the tube 212 so that it corresponds to the oronasoparynx (see FIG. 6). Once inflated, as shown in FIG. 6, the device 210 may be gently pulled back such that the second cuff 240 will be positioned in the opening in the nasopharynx NP and will prevent dislodgement of the tube 212 back out of the nose N and the cuff 214 from the hypopharynx to oropharynx. The second inflatable cuff 240 may be coupled to the inflation passageway 216 along with the inflatable cuff 214 to provide concurrent inflation and deflation of both cuffs. Alternatively, a second inflation passage (not shown) may be provided extending along the airway tube 212 and coupled only to the second inflatable cuff 240 to allow inflation and deflation thereof separate from the inflation and deflation of the inflatable cuff 214.

The embodiments of the nasal airway management device may be useful and beneficial in various medical situations. For example, the device may be utilized for rescue ventilation in emergency situations to provide effective and reliable ventilation by nurses or other non-anesthesia trained personnel involved in the management of a coding patient before arrival of anesthesia or emergency rooms doctors and placement of an endotracheal tube. Additionally, the device may be utilized in the early postoperative period to prevent a patient's tongue and soft tissue from collapsing onto the pharynx and obstructing the patient's upper airway while also providing the ability to rapidly transition to PPV by inflating the cuff when a patient stops breathing due to over-sedation, muscle weakness or residual anesthetic effects.

The device may also be utilized in outpatient procedures requiring sedation. These procedures (e.g. colonoscopies, endoscopies, transesophageal echocardiograms) are done under anesthesia sedation with intravenous general anesthetics (e.g. propofol) or combination of narcotic with anxiolitic. One of the anesthetic goals of such a technique is to keep patients breathing on their own (spontaneous ventilation) while supplemental oxygen is provided via nasal cannula. Emergency situations arise when a patient is over-sedated and stops breathing. Effective use of the device in place of conventional nasal cannulas will dramatically improve safety, by allowing timely, effective and smooth transition to PPV in a case of anesthetic overdose and cessation of spontaneous ventilation. Similar use under general anesthesia may be provided.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

We claim:

1. An airway management device, comprising:
an airway tube defining a lumen extending between a proximal end and a distal end, wherein at least the distal end of the airway tube is configured to be inserted through a patient's nasopharyngeal passageway;
an inflatable cuff attached at or near the distal end of the airway tube, wherein the inflatable cuff defines an opening fluidly coupled with the lumen of the airway tube; and
an inflation passage extending along the airway tube and coupled to the inflatable cuff to allow inflation and deflation thereof, wherein when in a deflated state the inflatable cuff is configured to be inserted through the patient's nasopharyngeal passageway, and wherein when in an inflated state the inflatable cuff is configured to expand to form a seal around the patient's supraglottic laryngeal inlet.

2. The airway management device of claim 1, wherein the inflatable cuff comprises a flexible material densely packed about the distal end of the airway tube when the inflatable cuff is in the deflated state to facilitate insertion of the distal end of the airway tube through the patient's nasopharyngeal passageway.

3. The airway management device of claim 2, wherein the densely packed inflatable cuff defines a substantially conical extension of the airway tube.

4. The airway management device of claim 2, wherein when the inflatable cuff is positioned in the patient's laryngopharynx in the deflated state, the device is configured to maintain patient airway patency and allow spontaneous ventilation via the opening.

5. The airway management device of claim 1, wherein when the inflatable cuff is positioned in the patient's laryngopharynx in the inflated state, the device is configured to allow artificial ventilation of the patient via the opening.

6. The airway management device of claim 5, wherein the proximal end of the airway tube comprises a connector portion configured to be coupled to a ventilator, a ventilation bag, or an anesthetic circuit, and wherein the artificial ventilation comprises positive pressure ventilation (PPV), continuous positive airway pressure (CPAP), or positive end expiratory pressure (PEEP).

7. The airway management device of claim 1, wherein the inflatable cuff comprises polyvinylchloride.

8. The airway management device of claim 1, wherein the airway tube comprises polyvinylchloride.

9. The airway management device of claim 1, wherein the airway tube comprises a curved shape.

10. The airway management device of claim 1, wherein the inflation passage comprises a pilot tube attached along an external surface of the airway tube.

11. The airway management device of claim 1, wherein the inflation passage comprises a pilot tube attached along an internal surface of the lumen of the airway tube.

12. The airway management device of claim 1, wherein at least part of the inflation passage is integrally formed within a wall of the airway tube.

13. The airway management device of claim 1, further comprising an adjustable annular flange moveably disposed on an outer periphery of the airway tube near the proximal end.

14. The airway management device of claim 1, further comprising an electronic measurement device disposed on the airway tube.

15. The airway management device of claim 14, wherein the electronic measurement device is embedded in a wall of the airway tube.

16. The airway management device of claim 14, wherein the electronic measurement device comprises a temperature measurement probe selected from the group consisting of: a thermocouple, a thermistor, and a resistance temperature detector (RTD).

17. The airway management device of claim 14, wherein the electronic measurement device comprises a pH probe or a CO2 probe.

18. The airway management device of claim 1, further comprising a second inflatable cuff attached to the airway tube between the proximal and distal ends, wherein the second inflatable cuff is configured to be inflated in the patient's oropharynx to secure the airway tube therein and prevent the airway tube from sliding back into the nasopharyngeal passageway.

19. The airway management device of claim 18, wherein the inflation passage is coupled to the second inflatable cuff to allow inflation and deflation thereof.

20. The airway management device of claim 18, further comprising a second inflation passage extending along the airway tube and coupled to the second inflatable cuff to allow inflation and deflation thereof separate from the inflation and deflation of the inflatable cuff.

21. The airway management device of claim 1, wherein when in a deflated state the inflatable cuff has an outer dimension substantially equal to or less than an outer dimension of the airway tube.

22. A method for managing a patient's airway, comprising utilizing the airway management device of claim 1.

23. A method for managing a patient's airway, comprising:
inserting the distal end of airway management device of claim 1 through the patient's nasopharyngeal passageway to a position in the laryngopharynx, wherein the inflatable cuff is in the deflated state.

24. The method of claim 23, further comprising
maintaining the patency of the patient's airway and allowing spontaneous ventilation via the opening when the cuff is in the deflated state.

25. The method of claim 23, wherein the inflatable cuff comprises a flexible material densely packed about the distal end of the airway tube when the inflatable cuff is in the deflated state to facilitate insertion of the distal end of the airway tube through the patient's nasopharyngeal passageway.

26. The method of claim 23, further comprising
inflating the cuff until the supraglottic laryngeal inlet is substantially sealed; and
artificial ventilating the patient via the opening.

27. An airway management device, comprising:
means for providing a nasopharyngeal airway;
means for sealing a patient's supraglottic laryngeal inlet to allow artificial ventilation of the patient via the airway means; and
means for inflating and deflating the sealing means.
